(19) United States
(12) Patent Application Publication
(10) Pub. No.: US 2016/0206291 A1
(43) Pub. Date: Jul. 21, 2016

(54) LIVE ULTRASOUND IMAGE AND HISTORICAL ULTRASOUND IMAGE FRAME OVERLAPPING

(71) Applicant: General Electric Company, Schenectady, NY (US)

(72) Inventors: Jiajiu Yang, Wuxi (CN); Dongqing Chen, New Berlin, WI (US); Menachem Halmann, Bayside, WI (US); Eunji Kang, Brookfield, WI (US); Bo Dan, Wuxi (CN); Ye Wang, Wuxi (CN)

(21) Appl. No.: 14/599,456

(22) Filed: Jan. 16, 2015

(51) Int. Cl. A61B 8/08; A61B 8/00

(52) U.S. Cl. CPC A61B 8/5253; A61B 8/463; A61B 8/5207

(57) ABSTRACT

A method and apparatus present a live ultrasound image on a display of an ultrasound imaging system, retrieve a historical ultrasound image frame; and overlap at least portions of the historical ultrasound image frame with the live ultrasound image. A method and apparatus retrieve a historical ultrasound image frame, retrieve stored coordinates of a historical region of interest in the historical ultrasound image frame, display a selected ultrasound image frame from a live ultrasound image and display a current region of interest in the selected ultrasound image frame, the current region of interest having coordinates in the selected ultrasound image frame based upon the stored coordinates retrieved from the historical region of interest in the historical ultrasound image frame.

### BACKGROUND

Ultrasound images are often used to diagnose injuries or diseases. Different ultrasound images taken at different times are often used to determine how the injuries or diseases are changing. Obtaining accurate comparisons of the different ultrasound images is often tedious, time-consuming and prone to error.

### BRIEF DESCRIPTION OF THE DRAWINGS

FIG. 1 is a schematic diagram of an example ultrasound imaging system.

FIG. 2 is a flow diagram of an example method in which a historical ultrasound image frame and a live ultrasound image are overlapped.

FIG. 3 is a schematic diagram illustrating one example overlapping of a historical ultrasound image frame with respect to a live ultrasound image.

FIG. 4 is a schematic diagram illustrating another example overlapping of a historical ultrasound image frame with respect to a live ultrasound image.

FIG. 5 is a schematic diagram of an example side-by-side display of a historical ultrasound image frame and a live ultrasound image.

FIG. 6 is a schematic diagram of a side-by-sigh display of the historical ultrasound image frame and the live ultrasound image being overlapped by the historical ultrasound image frame.

FIG. 7 is a perspective view of another ultrasound imaging system, an example implementation of the imaging system of FIG. 1.

FIG. 8 is a flow diagram of another example method for capturing and analyzing ultrasound information.

FIG. 9 is an example screenshot of an example side-by-side display of a historical ultrasound image frame and a live ultrasound image.

FIG. 10 is an example screenshot of an example side-by-side display of another historical ultrasound image frame and another live ultrasound image being overlapped by the historical ultrasound image frame.

FIG. 11 is an example screenshot of an example side-by-side display of another historical ultrasound image frame and another live ultrasound image being overlapped by the historical ultrasound image frame at first skeletal and background display settings.

FIG. 12 is an example screenshot of an example side-by-side display of the historical ultrasound image frame and the live ultrasound image of FIG. 11 being overlapped by the historical ultrasound image frame at second skeletal and background display settings.

FIGS. 13-16 are example screenshots illustrating the historical ultrasound image frame and the live ultrasound image of FIG. 10 with their scan planes being progressively moved into alignment with one another.

FIG. 17 is an example screenshot of an example side-by-side display of a historical ultrasound image frame and a live ultrasound image without depiction of blood flow overlapped by portions of the historical ultrasound image frame.

FIG. 18 is an example screenshot of an example side-by-side display of the historical ultrasound image frame of FIG. 17 and the live ultrasound image of FIG. 17 with reinstated depiction of blood flow.

FIG. 19 is an example screenshot of an example side-by-side display of another historical ultrasound image frame with a region of interest for which measurement functions were performed and for which results are displayed and a current ultrasound image frame with the same region of interest and having the same measurement function results displayed.

### DETAILED DESCRIPTION OF EXAMPLES

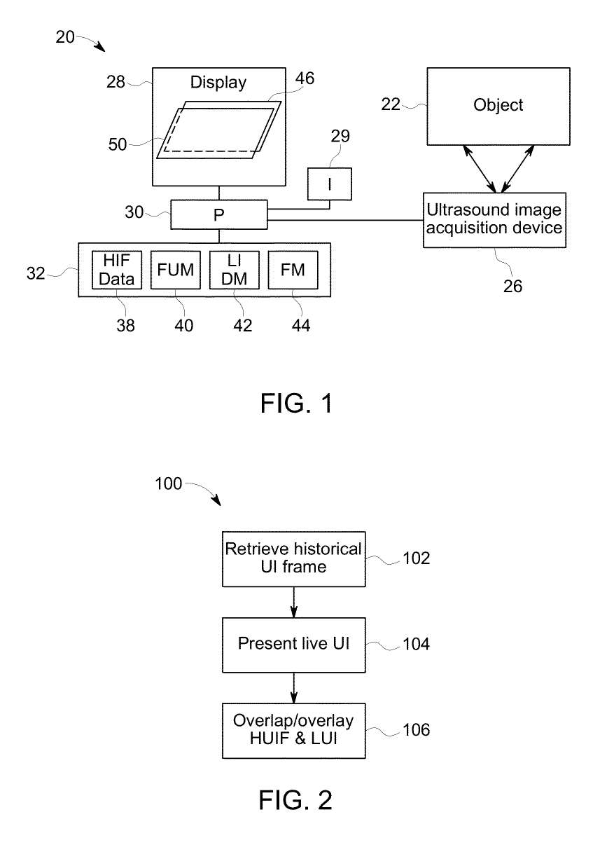

FIG. 1 schematically illustrates an example ultrasound imaging system 20. Ultrasound imaging system 20 facilitates enhanced alignment of a live ultrasound scan plane of an object 22 with an scan plane of a prior or historical ultrasound image frame of the object or other objects. Once approved alignment of an scan plane of the live ultrasound image and the scan plane of the historical ultrasound image frame have been achieved, current ultrasound image frames are captured from the live ultrasound image for comparison with the historical ultrasound image frame. As will be described hereafter, ultrasound imaging system 20 further facilitates more reliable and accurate comparison between the current ultrasound image frame and the prior captured ultrasound image frame. For example, in one implementation, ultrasound imaging system 20 facilitates the capture of a current ultrasound image frame illustrating inflammation symptoms associated with rheumatoid arthritis of joints and further facilitates comparison of the current ultrasound image frame with historical ultrasound image frames of the joints to facilitate accurate diagnosis.

Ultrasound imaging system 20 comprises ultrasound image acquisition device 26, display 28, input 29, processor 30 and memory 32. Ultrasound image acquisition device 26 comprises a device by which ultrasound (ultrasonic) waves or pulses are directed into object 22, such as the anatomy of a person or animal, and by which reflections of such waves are sensed to produce signals. In one implementation, the ultrasound image acquisition device comprises a transducer having quartz crystals, piezoelectric crystals, that change shape in response to application of electrical currents so as to produce vibrations or sound waves. Likewise, the impact a sound or pressure waves upon such crystals produces electrical currents. As a result, such crystals send and receive sound waves. In one implementation, ultrasound acquisition device 26 comprises ultrasound scanning device in which the transducer is mechanically positioned with respect to object 22. In another implementation, ultrasound acquisition device 26 comprises a manually positioned device, such as a hand-held probe. In one implementation, the probe may be positioned against the exterior of an anatomy or object being imaged. In another implementation, the probe may be partially inserted into the anatomy or object. Signals output by ultrasound image acquisition device 26 are transmitted to processor 30 for the generation display of images on display 28.

Display 28 comprises a screen or other display by which the results from ultrasound image acquisition device 26 are visibly presented to a caretaker, such as a doctor or nurse. In one implementation, display 28 comprises a single monitor or screen associated with processor 30 or in communication with processor 30. In another implementation, display 20 comprises multiple screens under the control of processor 30.

Input 29 comprises one or more devices by which a user may enter inputs, commands or selections to system 20. In one implementation, input 29 comprises a keyboard. In another implementation, input 29 comprises switches, slider bars, pushbuttons, a keypad, a touchpad, a mouse, a microphone with associated speech recognition software, a stylus or touchscreen capabilities associated with display 28. Ibid. 29 facilitates the entry of data as well as the input of selections or commands selecting modes of operation and indicating when to enter and exit different modes of operation, such as a fusion mode as will be described hereafter.

Processor 30 comprises one or more processing units which control presentation of ultrasound images upon display 28. In one implementation, processor 30 additionally generates the ultrasound images using signals received from ultrasound image acquisition device 26. For purposes of this application, the term “processing unit” shall mean a presently developed or future developed processing unit that executes sequences of instructions contained in a memory 32. In the example illustrated, memory 32 comprises a non-transient or non-transitory computer-readable medium containing computer code for the direction of controller processor 30. Execution of the sequences of instructions causes the processing unit comprising processor 30 to perform steps such as generating control signals. The instructions may be loaded in a random access memory (RAM) for execution by the processing unit from a read only memory (ROM), a mass storage device, or some other persistent storage. In other embodiments, hard wired circuitry may be used in place of or in combination with software instructions to implement the functions described. For example, processor 30 may be embodied as part of one or more application-specific integrated circuits (ASICs). Unless otherwise specifically noted, the controller is not limited to any specific combination of hardware circuitry and software, nor to any particular source for the instructions executed by the processing unit.

As schematically illustrated by FIG. 1, memory 32 comprises historical image frame data section or storage 38, follow-up module 40, live image display module 40 and fusion module 44. Historical image frame data section or storage 38 comprises data or files associated with historical ultrasound image frames, each historical ultrasound image frame comprising a previously captured individual or static ultrasound image frame. In one implementation, the historical image frame data section or storage 38 comprises B-mode ultrasound image frames. In other implementations, the historical ultrasound image frames may comprise other image formats depicting additional information.

Such historical image frame data 38 comprises ultrasound image frames captured from one or more previous live ultrasound images at a prior imaging or scanning session. For example, such historical image frame data may have been captured hours, days, weeks, months or even years prior to the present time at which the live ultrasound image 46 is being taken. In one implementation, each historical ultrasound image frame stored in data storage 38 has one or more files, or links to files, containing the scanning parameters or settings of ultrasound image acquisition device 26 (or a different ultrasound image acquisition device 26) that were utilized during the generation of the historical ultrasound image frame. In one implementation, each historical ultrasound image frame stored in data storage 38 additionally or alternatively comprises one or more files, or links to files, containing the previously selected or identified historical regions of interest and the particular measurement functions that were carried out with respect to the historic ultrasound image frame or the historical regions of interest in the historical ultrasound image frame.

Follow-up module 40, live image display module 40, and fusion module 44 each comprise software, code, integrated circuitry or other types of program logic that direct or control processor 30 in the concurrent overlapping display of a historical ultrasound image frame and live stream of ultrasound images. In the example illustrated, follow-up module 40, live image display module 40, and fusion module 44 cooperate to carry out the example method 100 set forth in FIG. 2.

As indicated by block 102 in FIG. 2, follow-up module 40 directs processor 30 to retrieve an individual historical ultrasound image frame from data storage 38. In one implementation, follow-up module 40 retrieves the historical image frame from a data storage or data store which is part of memory 32 local to processor 30. In another implementation, follow-up module 40 retrieves the historical image frame from a remote memory, such as from a remote database or remote server, across a local area network or wide area network, such as the Internet. In one implementation, follow-up module 40 retrieves multiple historical ultrasound image frames or retrieves multiple links to multiple historical ultrasound image frames, wherein the files or links are displayed on display 28 for selection by a physician, nurse or practitioner through a touchscreen, manipulation of a cursor, speech recognition or the like.

In one implementation, such image frames comprise historical ultrasound image frames of the same object or same patient being examined, whether such image frames have been captured and stored days, weeks or months prior to the current exam. In yet other implementations, the retrieved historical ultrasound image frame comprises a model or a standard image frame to be used for comparison with the live ultrasound image or individual frames captured from the live ultrasound image. For example, in one implementation, the historical ultrasound image frame comprises an ultrasound image frame of a healthy individual, a healthy anatomy or the like of the same object currently being examined, from another real object or anatomy or from a generated hypothetical model of the anatomy.

Follow-up module 40 retrieves or extracts, from the files associated with the retrieved ultrasound image frame, the scanning or imaging parameters previously used by the ultrasound image acquisition device 26 (or another ultrasound image acquisition device) when the retrieved ultrasound image frame was generated or captured. As will be described hereafter with respect to FIGS. 5 and 6, in one user selected mode of operation, follow-up module 40 additionally directs processor 30 to display the retrieved historical ultrasound image frame for viewing, either by itself on a single screen or side-by-side (as seen in FIGS. 5 and 6) with the live ultrasound image for comparison.

As indicated by block 104 in FIG. 2, live image display module 42 directs processor 30 to output control signals causing display 28 to present a live or real time ultrasound image 46 based upon signals received from ultrasound image acquisition device 26. The live ultrasound image presented on display 28 comprises a series of ultrasound image frames presented at a high frequency in real time. The live ultrasound image presented on display 28 by module 42 is a result of the live stream of ultrasound image signals received from ultrasound image acquisition device 26. Movement of object 22 or movement of the ultrasound image acquisition device 26 causes movement of the live ultrasound image 46 such that different scan planes are presented as device 26 and/or object 22 are moved relative to one another.

In one implementation, the live ultrasound image 46 comprises a B-mode image. In another implementation, the live ultrasound image 46 comprises a color flow image, a power Doppler image (PDI) or a high resolution PDI image. As will be described hereafter, in some implementations, the live ultrasound image presented on display 28 is further modified by fusion module 44 to enhance viewing of the overlapped live ultrasound image and historical ultrasound image frame. In yet other implementations, the live ultrasound image 46 may comprise other imaging formats or modes.

In the example illustrated, live image display module 42 further adjusts or controls the operational settings or scanning parameters of ultrasound image acquisition device 26. In one implementation, live image display module 42 automatically receives the retrieved scanning parameters associated with the retrieved historical ultrasound image frame and automatically controls ultrasound image acquisition device 26 based upon the prior retrieved scanning parameters previously used when the historical ultrasound image frame was generated. In one implementation, live image display module 42 automatically utilizes the same scanning parameters associated with the retrieved historical ultrasound image frame for the generation of the live ultrasound image. In another implementation, live image display module 42 performs adjustments or modifications upon the prior scanning parameters.

In yet other implementations, live image display module 42 prompts for and receives input or manual entry of the scanning parameters for the ultrasound image acquisition device 26 to generate the live ultrasound image. In one such implementation, live image display 42 displays and suggests use of the prior scanning parameters associated with the historical ultrasound image frame. In such an implementation, the user may enter or input the exact same prior scanning parameters or may make adjustments to the prior scanning parameters.

As indicated by block 106 of FIG. 2, fusion module 44 directs processor 30 to “fuse” the live ultrasound image with the historical ultrasound image frame. Fusion module 42 outputs control signals causing display 28 to present the retrieved historical ultrasound image frame 50 in an overlapping or overlaid relationship with respect to the live ultrasound image 46 being displayed on display 28. In the example illustrated, the overlapping live image or the overlapping historical image frame is outlined or is made at least semi-transparent such that the underlying live image or the underlying historical image frame is viewable or discernible through the overlying live image or the historical image frame. In the example illustrated in FIG. 1, the historical image frame is placed on top of or in overlapping relationship with the underlying live image.

Because the underlying image, whether it be live image 46 or the historical ultrasound image frame 50, is viewable through the overlying image, the user may reposition either object 22 and/or the ultrasound image acquisition device 26 relative to one another to reposition the scan plane of the live image 46 until the scan plane of the live image 46 has sufficient alignment with the historical ultrasound image frame 50. In one implementation, such alignment is determined by the user of system 20 based upon the user's perception of alignment. For example, the ultrasound system 20 may align the underlying scan plane of the live image 46/historical ultrasound image frame 50 and the overlying scan plane of the live image 46/historical ultrasound image frame 50 by aligning distinctive anatomical features or landmarks, such as skeletal structures/bones or muscle/tissue. Once the ultrasound image acquisition device 26 and object 22 are appropriately positioned relative to one another such that the scan planes of the live ultrasound image 46 and of the historic ultrasound image frame 50 are in user approved, sufficient alignment, at least one individual current image frame of the stream of image frames forming the live ultrasound image 46 may be frozen or captured for direct subsequent comparison and analysis with respect to the historical ultrasound image frame 50. Because the current ultrasound image frame being compared with the historical ultrasound image frame has substantially the same scan plane, the results of the comparison are more accurate and valid.

In one implementation, in addition to overlapping the live ultrasound image and the historic ultrasound image frame, fusion module 44 additionally carries out modifications upon one or both of the live ultrasound image and the historical ultrasound image frame to facilitate user manipulated alignment of the live ultrasound image and the historical ultrasound image frame. For example, in one implementation, the live ultrasound image, depending upon its format, may include blood flow data or color flow data. In such an implementation, when in the fusion mode in which the live image and the historical image are being overlapped, fusion module 44 automatically modifies the depiction of the blood flow or color flow in the live ultrasound image. For example, in one implementation, fusion module 44 completely removes the blood flow or color flow pixels depicting blood flow. For purposes of this disclosure, “removal” of such pixels encompasses making such textiles completely transparent, removal of such pixels and replacement of such pixels with other pixels, or changing the color or other characteristics of such pixels such that they are not distinguishable from surrounding pixels. In one implementation, fusion module 44 maintains such color flow pixels, but reduces their visibility in the live ultrasound image.

In one implementation, fusion module 44 additionally or alternatively modifies portions of the historical image frame being overlapped with the live ultrasound image to facilitate discernment between the live ultrasound image and the historical ultrasound image frame. For example, in one implementation, fusion module 44 applies one or more colors to the entire historical ultrasound image frame or features of the historical ultrasound image frame, wherein the color or colors are different from the color colors associated with the live ultrasound image. In another implementation, fusion module 44 modifies the line thickness, brightness, intensity, flashing frequency or the like of the entire historical ultrasound image frame or features of the historical ultrasound image frame so as to visibly distinguish the historical ultrasound image frame with respect to the live ultrasound image.

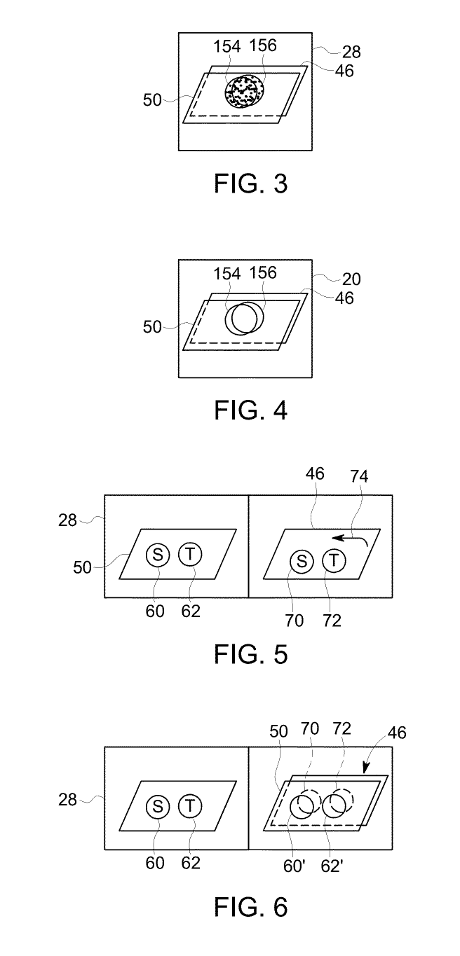

As noted above, fusion module 46 presents the live ultrasound image 46 and the historical ultrasound image frame 50 in an overlapping or overlaying relationship, wherein the underlying live image 46 or the underlying historical image frame 50 are viewable or discernible through the overlying live image 46 or the overlying historical image frame 50. FIGS. 3 and 4 schematically illustrate an implementation where the live ultrasound image 46, the stream of high-frequency frames forming the live image, are displayed beneath the overlying historical ultrasound image frame 50. As shown by FIGS. 3 and 4, in the example illustrated, system 20 is operable in two user selectable modes. In the first mode shown in FIG. 3, anatomical structures 154, such as muscle, tissue, skeletal or bone, of the overlying historical ultrasound image frame 50 are semi-transparently displayed such that the anatomical structures 156 of the ever-changing live ultrasound image 46 may viewed beneath or through such anatomical structures 154. In the second mode shown in FIG. 4, perimeters, boundaries or edges of identified anatomical structures 154 are presented in solid lines while the interiors of such boundaries or edges are transparent (not illustrated) or semi-transparent. In the second mode of operation, outlines of anatomical structures 154 of the historical ultrasound image 50 are presented, allowing the anatomical structures 156 of the underlying, ever-changing live ultrasound image 46 are viewable through or within the outline of the overlying anatomical structures 154.

In yet additional user selectable modes of operation, system 20 further visibly distinguishes at least one of the overlying anatomical structure 154 or the underlying anatomic structure 156. In one implementation, fusion module 44 directs processor 30 to highlight one of the underlying or overlying anatomical structures. In one implementation, fusion module 44 directs processor 30 to highlight the overlying anatomical structure or structures 154 of the historical ultrasound image 50. In one implementation, such highlighting is performed by providing the overlying anatomical structure or structures 154 with a color, shade or brightness distinct from that of the color, shade or brightness of the underlying anatomical structures 156 of the live ultrasound image 46. In another mode, such highlighting is achieved by presenting or displaying the overlying anatomical structures 154 such that the overlying anatomical structures 154 flash or changing color, shade or brightness at a frequency distinct from the underlying live image 46. In yet another user selectable mode of operation, such highlighting is achieved by identifying edges, boundaries or an outline of the overlying anatomic structure 154 and displaying the identified outline, boundary or edges with a color, shade, brightness, line thickness or display frequency different than that of the underlying anatomical structure 156 of the real-time, live ultrasound image 46.

FIGS. 5 and 6 schematically illustrate yet another user selectable mode of operation for system 20. As shown by FIGS. 5 and 6, follow-up module 40 and live image display module 42 direct processor 30 to generate control signals presenting side-by-side ultrasound images on display 28, concurrently presenting the historical ultrasound image frame 50 and the live ultrasound image 46. Although the side-by-side images are illustrated as being concurrently presented on two adjacent portions of display 28, in other implementations, such side-by-side images are presented on multiple display monitors or screens.

FIG. 5 illustrates a comparison or follow-up mode in which the live ultrasound image 46 is presented, without overlap, alongside the historical ultrasound image frame 50. As shown by FIG. 5, follow-up module directs processor 32 present historical ultrasound image 50. Historical ultrasound image frame 50 comprises images or visible depictions of substantially stationary structures such as bones or skeletal structures 60 and muscle structures or tissue 62. The skeletal structures may be organic or may be inorganic, artificial. In some implementations, the historical ultrasound image frame 50 may additionally comprise images or depictions of other substantially stationary structures or objects such as implants amongst the tissue and bones. In one implementation, historical ultrasound image 50 comprise a B-mode image frame.

As further shown by FIG. 5, live ultrasound images lay module 42 concurrently displays live ultrasound image 46. Live ultrasound image 46 comprises skeletal structure 70, tissue 72 and depictions of dynamic features, such as blood flow 74. In one implementation, the live ultrasound image may comprise color flow data, PDI data or high resolution PDI data. As noted above, in one implementation, live ultrasound image display module 42 controls ultrasound image acquisition device 26 (shown in FIG. 1) such that the device 26 captures the live ultrasound image 46 using scanning parameters or settings based upon, if not the same as, the same scan parameters or settings that were used to capture the displayed historical ultrasound image frame 50.

FIG. 6 illustrates an example fusion mode. In the fusion mode shown FIG. 6, the historical ultrasound image 50, shown on the left side of display 28, is copied over and overlapped upon the live ultrasound image 46 on the right side of display 28 to form skeletal structures 60′ and tissue 62′. Those portions of the historical sun image frame 50 that are copied over and overlaid upon the underlying live image 46 are modified or otherwise presented so as to permit viewing of at least portions of the overlap and underlying live ultrasound image 46. As noted above, in one implementation, skeletal structure 60′ and tissue 62′ corresponding to structure 60 and tissue 62, respectively, of the historical sun image 50 are either outlined or made semi-transparent.

As shown by the right side of display 28 in FIG. 6, fusion module 44 continues to display or present the skeletal structures 70 and the tissue 72 in the live ultrasound image, but removes or at least diminishes the dynamic features, such as blood flow 74 (shown in FIG. 5) in the live ultrasound image 46. In one implementation, fusion module 44 additionally highlights one or both of skeletal structures 60′ and tissue 62′ of the historical ultrasound image frame 50 when being overlapped with the live ultrasound image 46. As described above, in one implementation, fusion module 44 applies one or more colors to skeletal structure 60′ and/or tissue 62′ of the historical ultrasound image frame 50 and/or modifies the intensity, brightness, pixel size, line width or the like of skeletal structure 60′ and/or tissue 62′ of the historical ultrasound image frame 50. In one implementation, the historical ultrasound image frame presented on the left side of display 28 is not altered when in the fusion mode. In yet another implementation, the historical ultrasound image frame presented on the left side of display 28 is modified in a similar fashion to the copy of the historical sun image frame 50 that is overlapped upon the live ultrasound image when system 20 is in the fusion mode illustrated in FIG. 6.

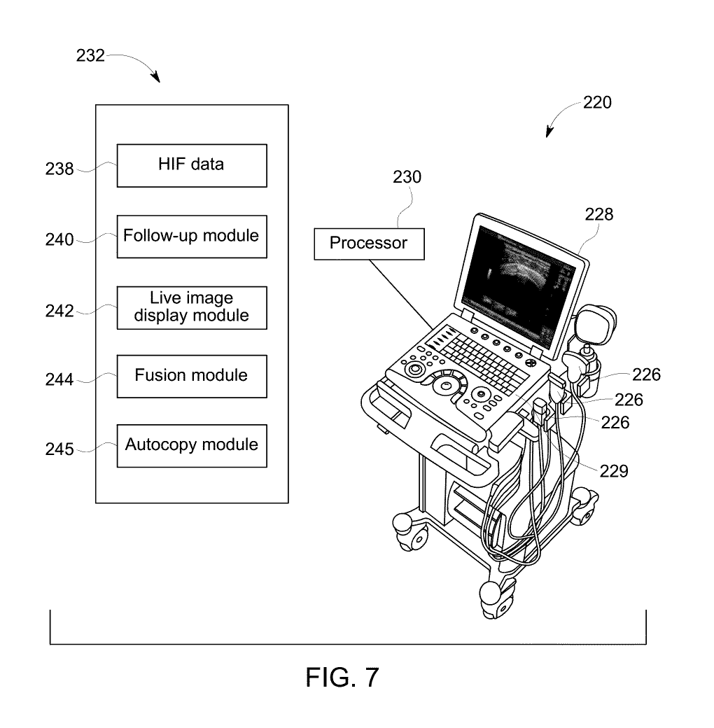

FIG. 7 illustrates ultrasound imaging system 220, an example implementation of ultrasound imaging system 20. Ultrasound imaging system 220 comprises ultrasound image acquisition devices 226, display 228, input 229, processor 230 and memory 232 comprising historical ultrasound image frame data storage 238, follow-up module 240, live image display module 242, fusion module 244 and auto copy module 246. Ultrasound image acquisition devices 226 comprise devices by which ultrasound (ultrasonic) waves or pulses are directed into an object or patient, such as the anatomy of a person or animal, and by which reflections of such waves are sentenced to produce signals. In one implementation, the ultrasound image acquisition device comprises a transducer having quartz crystals, piezoelectric crystals, that change shape in response to application of electrical currents so as to produce vibrations or sound waves. Likewise, the impact of sound or pressure waves upon such crystals produce a lot of currents. As a result, such crystals send and receive sound waves. In the example illustrated, ultrasound image acquisition device 226 comprise manually positionable ultrasound probes. In other implementations, ultrasound image acquisition device 226 comprises a mechanical scanner which mechanically moves in positions one or more ultrasound transducers relative to a patient. In one implementation, the probe may be positioned against the exterior of an anatomy or object being imaged. In another implementation, the probe may be partially inserted into the anatomy are object. Signals output by ultrasound image acquisition devices 226 are transmitted to processor 230 for the generation display of images on display 228.

Display 228 and input 229 are similar to display 28 and input 29, respectively, described above except that display 228 and input 229 comprise specific implementations of display 28 and input 29, respectively. Display 228 comprises a screen or other display by which the results from ultrasound image acquisition device 26 are visibly presented to a caretaker, such as a doctor or nurse. In the example illustrated, display 228 comprises a single monitor or screen associate with processor 230.

Input 229 comprises one or more devices by which a user may enter inputs, commands or selections to system 20. In the example illustrated, input 229 comprises a keyboard, various pushbuttons and a trackball. In another implementation, input 229 comprise other types of input devices such as other switches, slider bars, pushbuttons, a keypad, a touchpad, a mouse, a microphone with associated speech recognition software, a stylus or touchscreen capabilities associated with display 28. As with input 29, input 229 facilitates the entry of data as well as the input of selections or commands selecting modes of operation and indicating when to enter and exit different modes of operation, such as a fusion mode as will be described hereafter.

Processor 230 is similar to processor 30 described above. Processor 230 comprises one or more processing units which control presentation of ultrasound images upon display 28. In the example illustrated, processor 230 additionally generates the ultrasound images using signals received from the particular ultrasound image acquisition device 226 being used. Processor 230 performs analysis and generates control signals for the operation of device 226 as well as display 228 following instructions provided by modules 240, 242, 244 and 246 of memory 232.

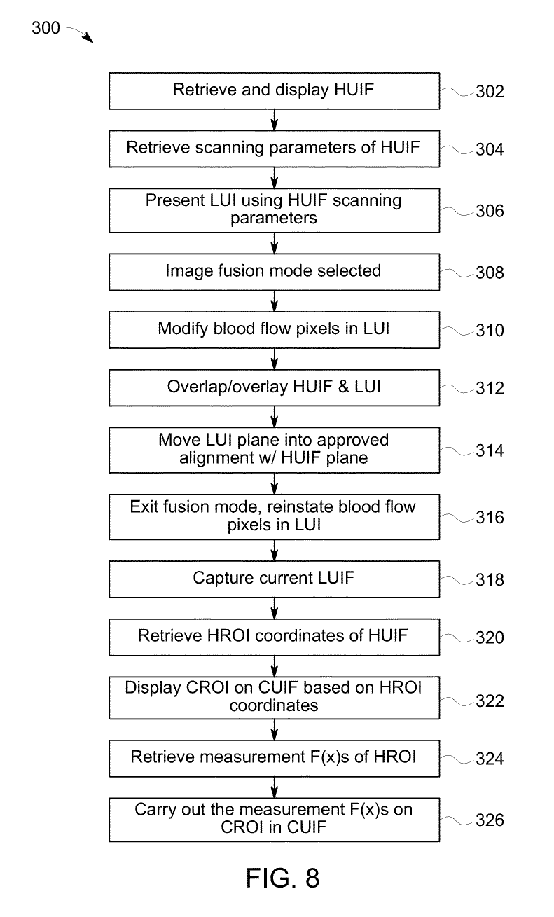

Historical ultrasound image frame data storage 238 is similar to historical ultrasound image frame data storage 38 described above. Follow-up module 240 and fusion module 244 utilize files or data from data storage 238. Follow-up module 240, live image display module 242, fusion module 244 and auto copy module 246 each comprise software, code, integrated circuitry or other program logic to direct processor 30. Follow-up module 240, live image display module 242, fusion module 244 and auto copy module 246 cooperate to carry out the example method 300 outlined in FIG. 8.

As indicated by block 302 in FIG. 8, follow-up module 240 directs processor 230 to retrieve and display an individual historical ultrasound image frame from data storage 238. In one implementation, follow-up module 240 retrieves the historical image frame from a data storage or data store which is part of memory 232 local to processor 230. In another implementation, follow-up module 40 retrieves the historical image frame from a remote memory, such as from a remote database or remote server, across a local area network or wide area network, such as the Internet. In one implementation, follow-up module 240 retrieves multiple historical ultrasound image frames or retrieves multiple links to multiple historical ultrasound image frames, wherein the files or links are displayed on display 228 for selection by a physician, nurse or practitioner through a touchscreen, manipulation of a cursor, speech recognition or the like.

In one implementation, such image frames comprise historical ultrasound image frames of the same object or same patient being examined, whether such image frames have been captured and stored days, weeks or months prior to the current exam. In yet other implementations, the retrieved historical ultrasound image frame comprises a model or a standard image frame to be used for comparison with the live ultrasound image or individual frames captured from the live ultrasound image. For example, in one implementation, the historical ultrasound image frame comprises an ultrasound image frame of a healthy individual, a healthy anatomy or the like of the same object currently being examined, from another real object or anatomy or from a generated hypothetical model of the anatomy.

As indicated by block 304 in FIG. 8, follow-up module 40 retrieves or extracts, from the files associated with the retrieved ultrasound image frame, the scanning or imaging parameters previously used by the ultrasound image acquisition device 226 when the retrieved ultrasound image frame was generated or captured. In one implementation, follow-up module 40 identifies on display screen 28, the type of ultrasound acquisition device or the model of the ultrasound acquisition device that was used to obtain the prior historical ultrasound image frame being displayed.

As indicated by block 306 in FIG. 8, live image display module 242 directs processor 30 to output control signals causing display 228 to present a live or real time ultrasound image based upon signals received from ultrasound image acquisition device 26. The live ultrasound image presented on display 228 comprises a series of ultrasound image frames presented at a high frequency in real time. The live ultrasound image presented on display 228 by module 242 is a result of the live stream of ultrasound image signals received from the ultrasound image acquisition device 226. Movement of the patient or movement of the ultrasound image acquisition device 226 causes movement of the live ultrasound image such that different scan planes are presented as device 226 and/or the patient or object are moved relative to one another.

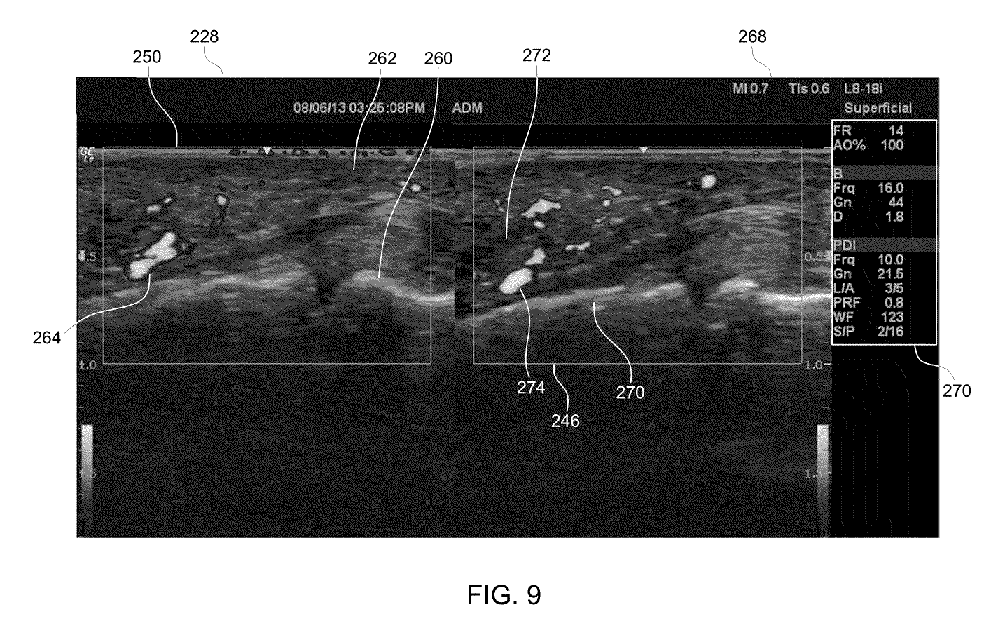

FIG. 9 is an example screenshot of display 228 illustrating the concurrent display of an example historical ultrasound image frame 250 on display 228 per block 302 of FIG. 8 alongside the live ultrasound image 246 on display 228 per block 306 of FIG. 8. As illustrated on the left side of FIG. 9, historical ultrasound image frame 250 is an individual image frame captured and stored at a prior time, such as hours, days, weeks, months or years prior to the current ongoing live ultrasound scanning producing the live ultrasound image 246. As shown by FIG. 9, historical ultrasound image frame 250 comprises skeletal features 260, tissue 262 and a still capture of blood flow 264, represented by color flow images.

As illustrated by the right side of FIG. 9, live ultrasound image 246 illustrates the dynamically changing depiction of skeletal features 260 and tissue 262 which change in response to relative movement between the ultrasound image acquisition device 26 being used in the patient. The live ultrasound image 246 further illustrates the dynamically changing depiction of blood flow 262. As a result, display 20 provides the user with a concurrent display of a static historical ultrasound image frame 250 and a dynamically changing live ultrasound image 246 which continuously changes during its presentation.

As further shown by FIG. 9, live image display module 42 provides additional information 268 such as the current date and time of the live image scan (08/06/13 03:25:08 PM), type or name of the ultrasound acquisition device currently being employed (L8-18i) and the type of scanning being done (superficial: Moscow skeletal superficial application, e.g. a hand or finger). Live image display module 42 further provides scanning parameter information. As noted above, live image display module 42 receives the scanning parameters of the historical ultrasound image frame 250 and utilizes the same scanning parameters for the current live scan or slightly modifies such prior scanning parameters for use with the live scan. In the example illustrated, such scanning parameters 270 utilized for the prior capture of ultrasound image frame 250 and the ongoing live ultrasound image 246 are presented on the right side of display screen 228. In the example illustrated, information regarding the B-mode ultrasound image (B) include ultrasound image frame rate (FR: 14), the acoustic power output (AO %: 100), the current mode of the ultrasound image (B: B Mode Ultrasound Image), the scanning frequency (Frq: 16.0 in (MHZ) currently chosen for the B Mode scan), the B mode ultrasound scanning game (Gn: 44), the B mode ultrasound image scanning depth (in cm) from the skin surface (D: 1.8). Information regarding the power Doppler imaging (PDI), used for musculoskeletal and rheumatology diagnosis comprises the scanning frequency used for the PDI mode (Frq: 10.0), the PDI gain (Gn: 21.5), the line density and frame average (L/A: 3/5), the pulse repetition frequency (in kHz) (PRF: 0.8), the wall filter used to differentiate tissue motion in real flow (WF: 123), and the spatial filter and packet size (S/P: 2/16). In other implementations, other scanning parameter information is presented or less than all of the illustrated scanning parameter information is presented.

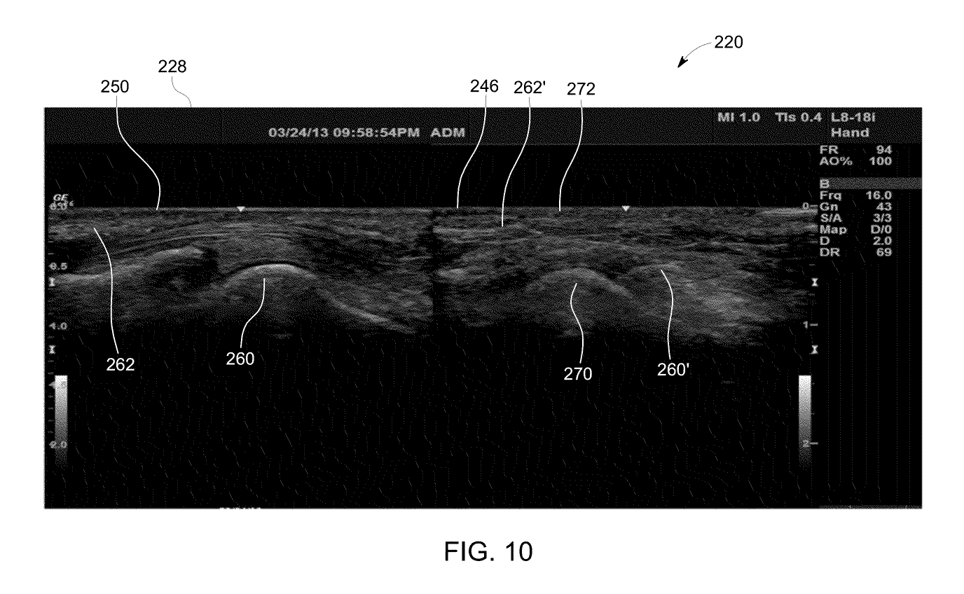

As indicated by block 308 in FIG. 8, upon receiving a command or selection via input 229, system 220 enters the fusion mode. As indicated by block 310 fusion module 244 modifies the pixels depicting blood flow 264 in the live ultrasound image 246 such that the blood flow pixels are less likely to obfuscate or interfere with user alignment of the live ultrasound image 246 and the historical ultrasound image frame 250. In the example illustrated, as illustrated by FIG. 10, fusion module 244 completely removes from the ongoing stream or live ultrasound image 246 those pixels that were previously colorized to represent blood flow 264. In the example illustrated, fusion module 244, without requiring further user input after switching to the fusion mode, automatically modifies or removes the pixels depicting blood flow 264 (shown in FIG. 9) in the live ultrasound image 246.

As further shown by FIG. 10, fusion module 244 additionally highlights those portions, images of skeletal structures 260′ and tissue 262′, of historical ultrasound image frame 250 that were copied and overlaid upon or on top of the dynamic live ultrasound image 246. In the example illustrated, fusion module 244 directs processor 230 to add a color (blue in the example) distinct from the color (black, gray, or the like) to each of such structures 260′ and tissue 262′ that are overlapped or overlaid upon ultrasound live image 246. In other implementations, system 220 provides prompts or other adjustable setting screens which allow a user to customize the color of such structures 260′ and/or 262′. Because those portions of historical ultrasound image 250 that are copied over are colorized, such portions are more visibly discernible from the live ongoing ultrasound image 246.

In one implementation, fusion module 244 automatically identifies, without user input or selection, anatomical landmarks in the historical image frame 250 and highlights such identified anatomical landmarks for use in aligning with the corresponding anatomical landmarks in the live ultrasound image frame 246. Examples of anatomical landmarks include distinct skeletal structures. In the example illustrated in FIG. 10, skeletal structures 260 serves as landmarks, wherein such identified landmarks are copied on top of the live ultrasound image 246 and highlighted with the color blue.

In the example illustrated, system 20 variably controls what anatomical landmarks, such as what skeletal structures, and what tissue features from a historical ultrasound image are highlighted in live ultrasound image such that they may be utilized as landmarks for alignment with corresponding landmarks in the live ultrasound image. For example, depending upon established opacity thresholds, some skeletal structures or some tissue features are highlighted (colorized in the example) in the live ultrasound image 246 while other skeletal structures or tissue features are not highlighted or colorized. In one implementation, a skeletal percentage value or setting controls or filters out what skeletal structures from the historical image frame 250, based upon a comparison of their normalized opacity in the historical image frame with respect to a normalized threshold value corresponding to the skeletal percentage value, are highlighted in the live image 246. A tissue or background percentage value or setting controls or filters out what tissue features from the historical image frame 250, based upon a comparison of their normalized opacity in the historical image frame relative to a normalized threshold value corresponding to the background percentage value, are highlighted in the live image 246. In one implementation, fusion module 44 applies default values for the skeletal percentage value and the background percentage value unless automatically adjusted based upon various image properties or adjusted manually by the user.

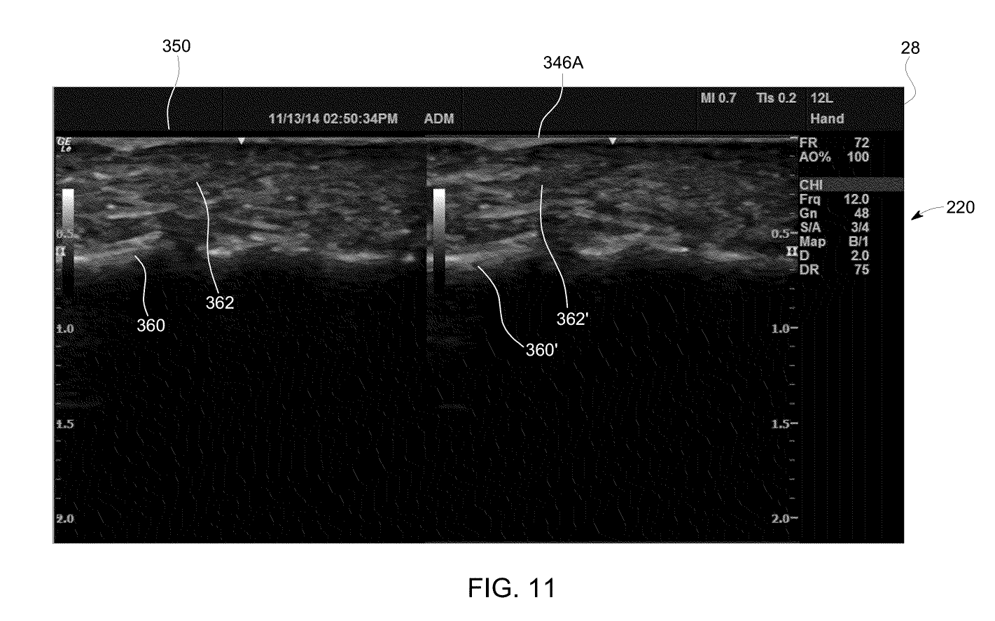

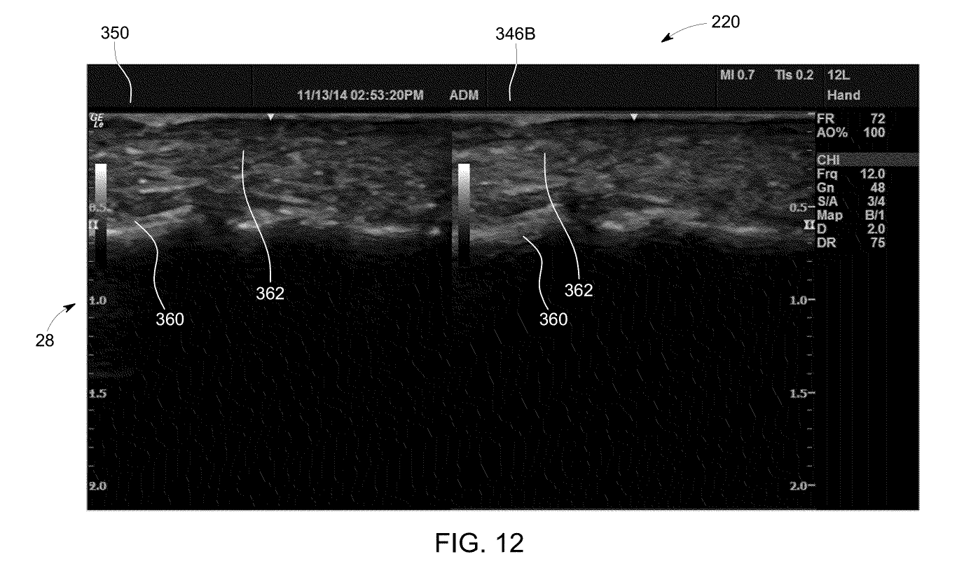

FIGS. 11 and 12 illustrate example screenshots illustrating the application of different skeletal and background percentage values to the same example historical ultrasound image frame 350 being overlaid upon two different live ultrasound images 346A and 346B having the same scan plane. In the example shown in FIG. 11, the background percentage value is 20% while the landmark or skeletal percentage value is 6%. In the example shown in FIG. 12, the background percentage setting or value is 30% while the skeletal percentage setting or value is 30%. As evident from a comparison of the live ultrasound images 346A and 346B in FIGS. 11 and 12, respectively, the lower background percentage value and the lower skeletal percentage value in FIG. 11 results in fewer portions or a lesser extent of the features of historical ultrasound image 350 being highlighted with color in live ultrasound image 346A. Conversely, the higher skeletal percentage setting and the higher tissue or background percentage setting in FIG. 12 results in a greater number of portions or a greater extent of the features of historical ultrasound image 350 being highlighted with color in live ultrasound image 346B.

As indicated by block 312 and FIG. 8, fusion module 244 overlaps or overlays portions of the historical ultrasound image frame, such as image frame 250 shown in FIG. 10, upon the live ultrasound image 246 shown in FIG. 10. As indicated by block 314 in FIG. 8, the scan plane of the live ultrasound image is moved into an approved level of alignment with the scan plane of the historical ultrasound image frame. Because portions of the historical ultrasound image frame 250 are not copied over onto the live ultrasound image 246 or are semi-transparent, the user may visibly discern the live ultrasound image frame 246 beneath or about those portions of the historical ultrasound image frame 250 overlapped by those portions of the historical ultrasound image frame 250. As a result, the user may reposition the ultrasound image acquisition device 26 relative to the patient to change the scan plane so as to sufficiently align the scan plane of the live ultrasound image 246 and its anatomical landmarks, skeletal structures and tissue features, with the corresponding anatomical landmarks of the scan plane of the historical image frame. As a result, system 220 allows a user to better align and match the scan plane of the live ultrasound image with the scan plane of the historical ultrasound image frame for more accurate comparisons with regard to what is seen in the historical ultrasound image frame and what is presently occurring or depicted in the live ultrasound image.

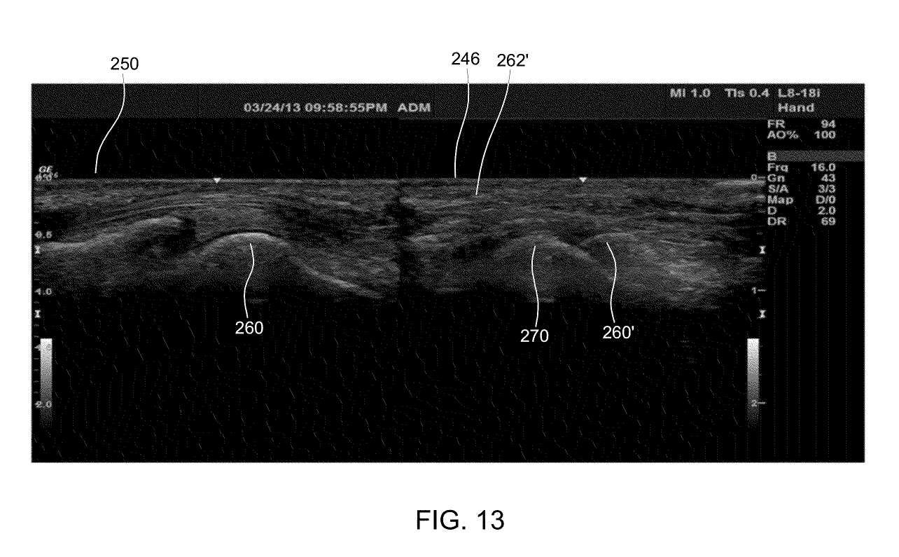

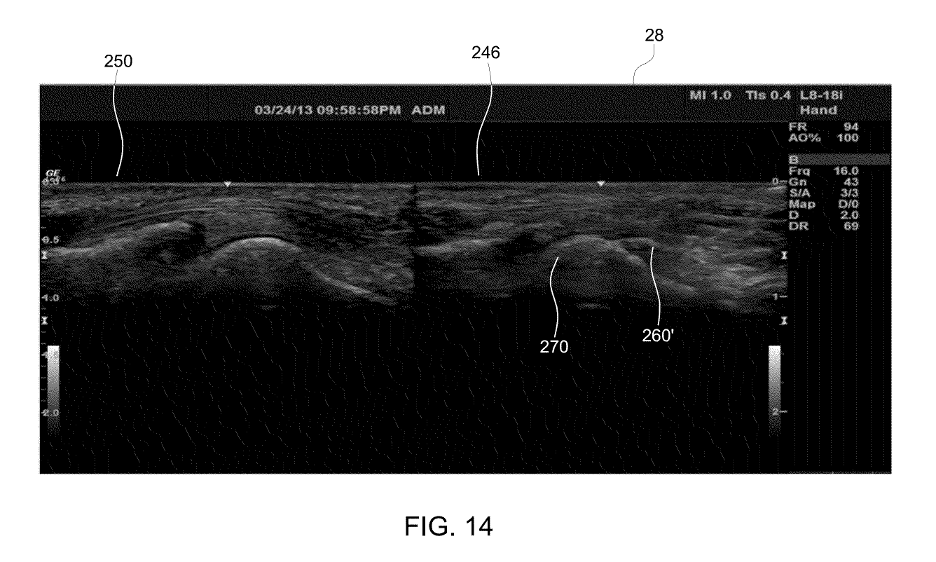

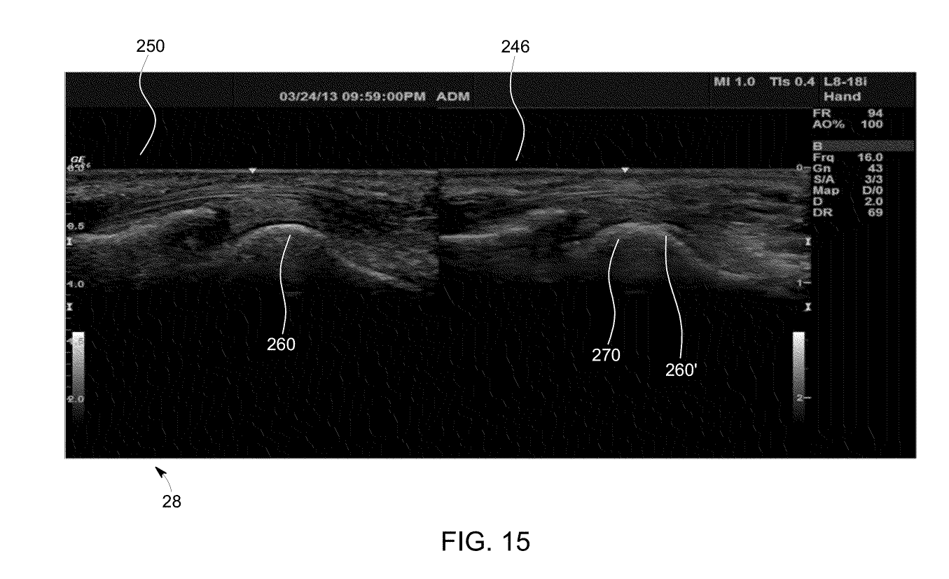

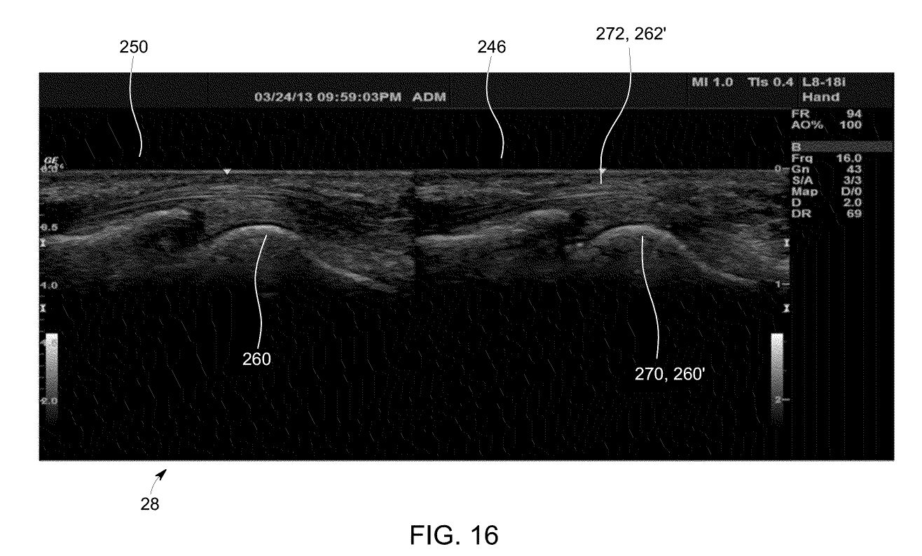

FIGS. 13-16 are example screenshots illustrating movement of the scan plane of the live ultrasound image 246—resulting from movement of the ultrasound acquisition device 226—into alignment a sufficient or approved level of alignment with the historical ultrasound image 250. As shown by FIG. 13, skeletal structures 270 depicted in the live ultrasound image 246 are initially to the left of their corresponding skeletal structures 260′ which are highlighted with a blue-collar. However, as shown by FIGS. 14-16, movement of the ultrasound image acquisition device 26 moves the scan plane to the right such that skeletal structures 270 are also moved to the right into alignment with skeletal structures 260′ (shown in FIG. 16). As shown by FIGS. 13-16, tissue features 262′ overlaid upon the live ultrasound image may also be used to facilitate alignment of the scan plane of the live ultrasound image 246 and the scan plane of the historical ultrasound image frame 250.

As indicated by block 316 in FIG. 8, once the scan plane of the live ultrasound image has been sufficiently aligned to the scan plane of the historical ultrasound image frame, the user may provide input via input 229 to exit the fusion mode. In the example illustrated, in response to receiving a command to exit the fusion mode, system 220 automatically reinstates the earlier presented pixels depicting blood flow in the live ultrasound image. In one implementation, when in the fusion mode, the live ultrasound image is a B mode image while the power Doppler image providing the color flow or blood flow pixels is not presented. Upon exiting the fusion mode, system 220 automatically reinstates the power Doppler imaging to once again depict the blood flow pixels.

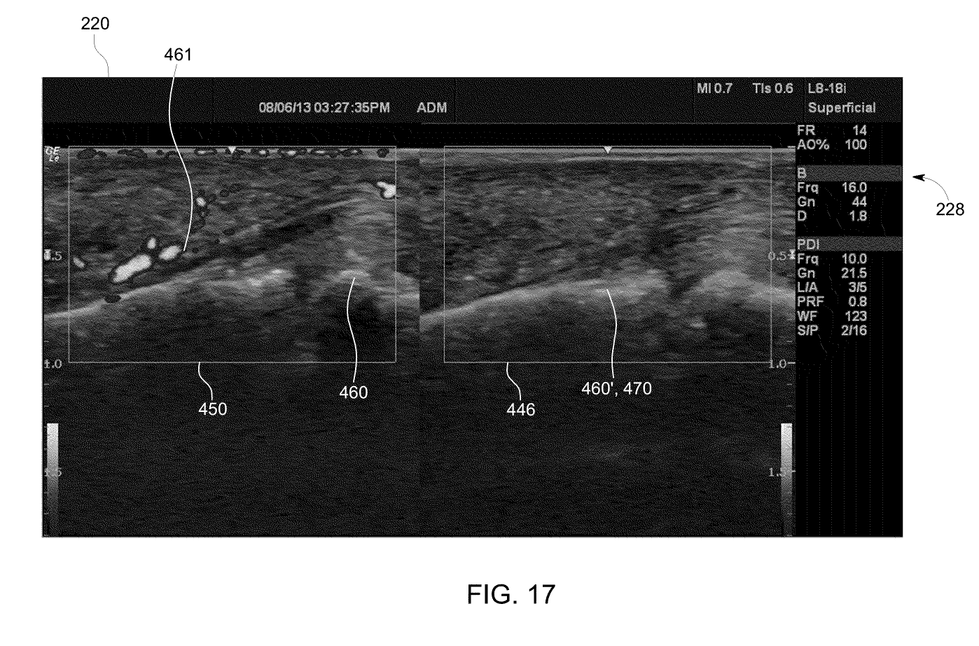

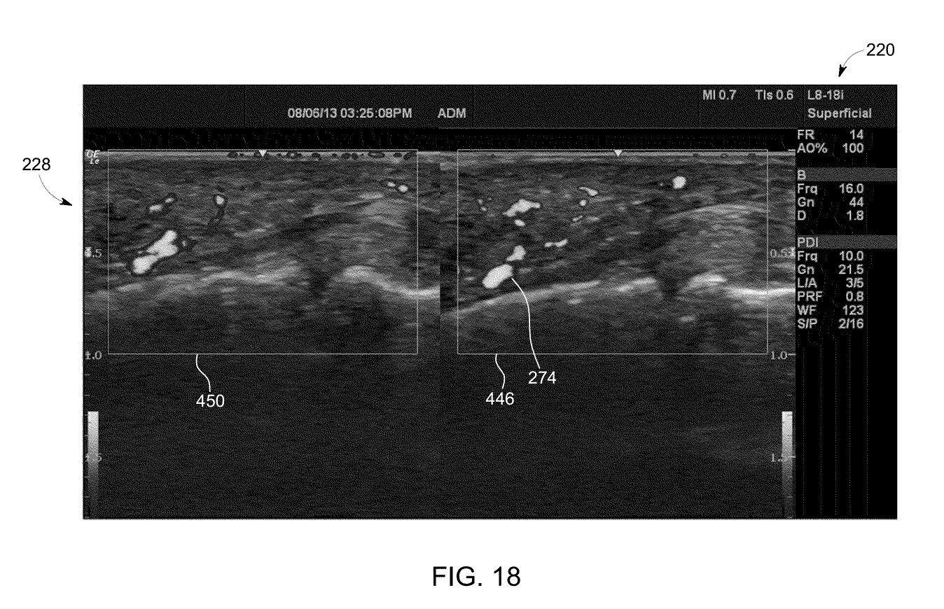

FIGS. 17 and 18 illustrate exiting from the fusion mode. FIG. 17 is a screenshot illustrating system 220 after the scan plane of example live ultrasound image 446 has been aligned with the scan plane of the example prior historical ultrasound image frame 450 to the satisfaction of the user. The left side of FIG. 17 depicts the historical ultrasound image frame 450 having skeletal structures 460 and further illustrating the captured state of blood flow 461 at the moment that the individual historical image frame 450 was captured. At the point in time depicted in FIG. 17, the example live ultrasound image 446 is the B mode image that was used for the prior fusion mode. FIG. 17 illustrate skeletal structures 460′ aligned with skeletal structures 470 of the live ultrasound image 446. As shown by FIG. 18, upon exiting the fusion mode, system 220 automatically reinstates the flow signals such that the pixels depicting blood flow are once again presented. In the example illustrated, the color flow mode or the power Doppler imaging mode is reinstated to once again depict blood flow 274.

As indicated by block 318 in FIG. 8, system 220 captures a current ultrasound image frame during the presentation of the live ultrasound image. As with the historical ultrasound image frames described above, the current ultrasound image frame is an individual frame or snapshot at a particular moment in time at a particular scan plane. Because the color flow mode or the power Doppler imaging mode representing the depicted blood flow is reinstated upon exit of the fusion mode, the snapshot forming the current ultrasound image frame includes depictions of blood flow at the picker moment in time. Such depictions of blood flow may then be utilized as part of the analysis and comparison with respect to the historical ultrasound image frame.

For example, in one implementation, the right side of display 228 in FIG. 18 may be captured, similar to a snapshot or screenshot, to form the current ultrasound image frame for comparison with the historical ultrasound image frame. In one implementation, the user, through input 229, selects the capture of the particular current ultrasound image frame from the live ultrasound image 446, preferably while the scan plane of the live ultrasound image 446 remains in substantial or satisfactory alignment with the scan plane of the prior historical ultrasound image frame 450.

Because system 220 facilitates more precise and accurate alignment of the scan planes of the historical ultrasound image frame and the current live ultrasound image frame, captured from a live ultrasound image, a more accurate comparison may be made between the two image frames. As shown by FIG. 7, to facilitate such analysis, system 220 additionally comprises auto copy module 245. Similar to fusion model 244, auto copy module 245 comprises software, code, integrated circuitry or other program logic stored in memory 238 to direct processor 230 to carry out functions. In the example illustrated, auto copy module 245 comprise a program logic to direct processor 230 to carry out the steps set forth in blocks 320-326 shown in FIG. 8. In other implementations, system to 20 omitted auto copy module 245.

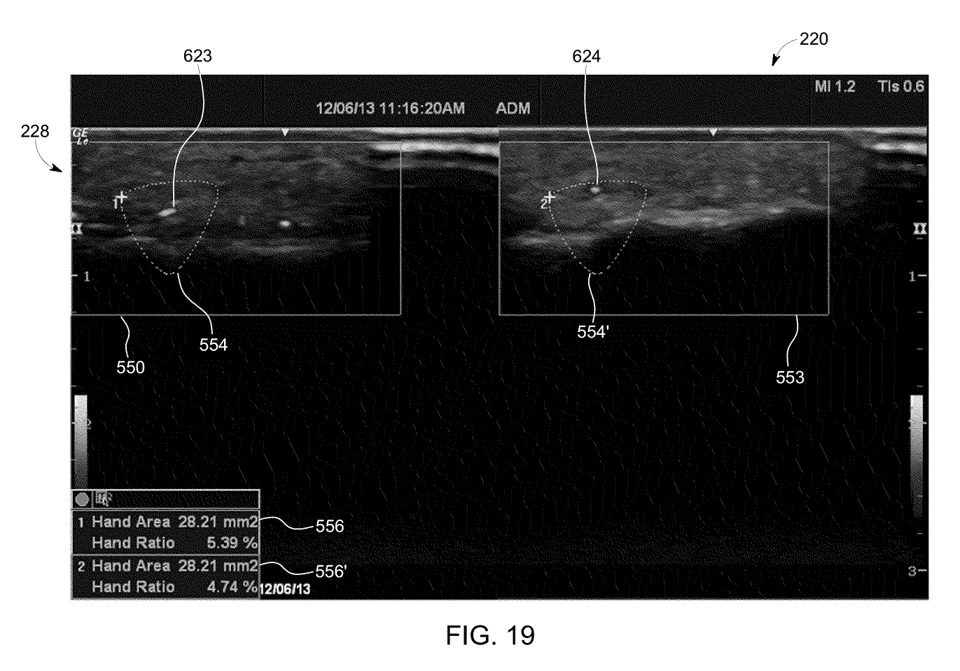

FIG. 19 illustrates an example screenshot of display 228 during one example use of auto copy module 245 according to blocks 320-326. As shown by FIG. 19, the left side of display 228 illustrates an example historical ultrasound image frame 550 while the left side of display 228 illustrates an example current ultrasound image frame 553 recently captured from a display live ultrasound image. In the example illustrated the historical ultrasound image frame and the current ultrasound image frame have substantially matching scan planes, achieved by operating system 220 in the fusion mode (described above) while aligning the scan plane of the live ultrasound image with the scan plane of the historical ultrasound image frame, prior to capturing the current ultrasound image frame 553.

The historical ultrasound image frame 550 depicts a previous historical region of interest 554 that was previously selected for analysis. The region of interest 554 identifies a particular defined region of the historical ultrasound image frame 550 for which the contents are analyzed. In the example illustrated, the region of interest 554 encompasses blood flow pixels 623. As indicated in the lower left-hand corner of display 228, system 220 displays the results 556 of the analysis performed on the region of interest 554. In the example illustrated, the ultrasound image frame is that of a hand. The region of interest has an area of 28.21 mm². In the example illustrated, the particular analysis on the contents of the region of interest 554 is to determine the ratio of depicted blood flow/inflammation (as represented by blood flow pixels 623) to the area of the hand in the particular region of interest 554. In the example illustrated, the ratio of blood flow/inflammation to the area of the hand in the region of interest is 5.39%.

As indicated by block 320 in FIG. 8, when an auto copy function or mode has been selected, auto copy module 245 retrieves information regarding the configuration, size and location of the region of interest in the historical ultrasound image frame 550. In the example illustrated, auto copy module 245 retrieves the locational coordinates of the historical ultrasound image frame region of interest 554. For example, auto copy module 254 retrieves the X and Y coordinates of each pixel forming the boundary of the region of interest 554 with respect to the historical ultrasound image frame being presented. In one implementation, such data is found in metadata associated with the historical ultrasound image frame 554 or is located in separate data files linked to the historical ultrasound image frame 550.

As indicated by block 322 in FIG. 8, auto copy module 245 utilizes the retrieved location and configuration information for the region of interest 554 from the historical ultrasound image 550 to generate and display a corresponding current region of interest 554′ in the current ultrasound image frame 553. The location as well as a configuration (size and shape) of the current region of interest 554′ is based upon the location and configuration (size and shape) of the historical region of interest 554. In one implementation, the current region of interest 554′ has substantially the exact same shape and the exact same size as the historical region of interest 554. In the example illustrated, both the historical region of interest 554 and the current region of interest 554′ have an area of 28.21 square mm. In such an implementation, the current region of interest 554′ also has pixels forming the boundary of the region of interest 554′, wherein the pixels have identical coordinates as compared to the corresponding pixels forming the boundary of historical region of interest 554. As a result, the regions of interest in both the historical image frame 550 and the current image frame 553 encompass substantially the precise same portions of the anatomy (portions of a hand in the illustrated example).

In other implementations, auto copy module 245 bases the configuration and location of the current region of interest 554′ upon the retrieved configuration and location values for the historical region of interest 554, wherein auto copy module 245 makes slight adjustments to one or more of the size, shape or location attributes of the current region interest 554′ with respect to the historical region of interest 554. Because the current region interest 554′ is automatically copied over or generated onto the current ultrasound image frame 553 by processor 230 under the direction of auto copy module 245 and based upon the historical region of interest in the previously stored and recorded historical ultrasound image frame 550, the same regions of interest in the same scan planes of the two image frames may be directly compared to achieve more accurate or reliable results.

As indicated by blocks 324 and 326, in one mode of operation, in lieu of having the user once again input those particular analytical functions or measurement functions that it be carried out for the contents of the region of interest or other characteristics of the region of interest, auto copy module 245 automatically retrieves the stored measurement functions that were applied to the region of interest 554 in the historical ultrasound image 550 and automatically carries out the same measurement functions for the current region of interest 554′ in the current ultrasound image frame 553. As a result, the user is not only presented with substantially the same relative region of interest, but is also automatically provided with the same measurement functions or analysis functions that were carried out for the historical ultrasound image frame 550.

As depicted in the lower left-hand corner of display 228, in the example illustrated, per block 324, auto copy module 245 retrieves metadata associated with historical ultrasound image frame 550 indicating that a measurement function comprising the determination of the percent of blood flow depicted in the region of interest was calculated upon the historical region of interest 554. Per block 326, auto copy module 245 carries out the same measurement function. In the example illustrated, auto copy module 245 has determined that, in the current ultrasound image frame 553, 4.74% of the area within the region of interest 554′, corresponding to the historical region of interest 554, is blood flow, as represented by blood flow pixels 624. The results 556′ are presented in the lower right-hand corner of display 228. In the example illustrated, within the region of interest, the present area of blood flow has dropped from 5.39% to 4.74%, indicating reduced inflammation.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. A method comprising:
presenting a live ultrasound image on a display of an ultrasound imaging system;
retrieving a historical ultrasound image frame; and
overlapping at least portions of the historical ultrasound image frame with the live ultrasound image.

2. The method of claim 1, wherein at least one of the live ultrasound image and the portions of the historical ultrasound image frame that are being overlapped are outlined or semi-transparent.

3. The method of claim 1 further comprising identifying anatomical landmarks in the historical ultrasound image frame, wherein the portions of the historical ultrasound image frame that are overlapped with the live ultrasound image comprise the identified anatomical landmarks.

4. The method of claim 1 further comprising highlighting the portions of the historical ultrasound image frame that overlap with the current ultrasound image.

5. The method of claim 3, wherein the portions are highlighted based upon color of the portions.

6. The method of claim 1 further comprising moving and changing the live ultrasound image being displayed relative to the overlapping portions of the prior ultrasound image frame in response to movement of an ultrasound probe.

7. The method of claim 1, further comprising removing portions of the live ultrasound image being overlapped by the historical ultrasound image frame.

8. The method of claim 7, wherein the portions of the live ultrasound image that are removed comprise pixels depicting blood flow.

9. The method of claim 1, wherein the ultrasound imaging system is operable in a first mode and a second mode and wherein the method further comprises:
displaying the live ultrasound image including pixels depicting blood flow when in the first mode;
while the live ultrasound image including pixels depicting blood flow is being displayed in the first mode, receiving first user input requesting the second mode of operation, and in response to receipt of the first user input, automatically (A) removing the pixels in the live ultrasound image depicting blood flow and (B) overlapping the historical ultrasound image frame with the live ultrasound image that is without the pixels depicting blood flow; and
while overlapping of the historical ultrasound image frame with the live ultrasound image is displayed in the second mode, receiving second user input requesting the first mode of operation, and in response to receipt of the second user input, automatically (A) displaying the live ultrasound image without the historical ultrasound image frame and with reinstated pixels indicating blood flow.

10. The method of claim 1 further comprising:
retrieving stored coordinates of a historical region of interest in the historical ultrasound image frame;
displaying a selected ultrasound image frame from the live ultrasound image; and
displaying a current region of interest in the selected ultrasound image frame, the current region of interest having coordinates in the selected ultrasound image frame based upon the stored coordinates retrieved from the historical region of interest in the historical ultrasound image frame.

11. The method of claim 10 further comprising:
moving at least one of an ultrasound probe and a patient relative to one another to change an scan plane of the live ultrasound image to substantially align the scan plane of the live ultrasound image with an scan plane of the historical ultrasound image frame; and
capturing a frame of the live ultrasound image when the scan plane of the live ultrasound image is substantially aligned with the scan plane of the historical ultrasound image, the captured frame constituting the selected ultrasound image frame.

12. The method of claim 10 further comprising:
retrieving stored settings indicating measurement functions associated with the historical region of interest of the historical ultrasound image frame; and
carrying out the measurement functions with respect to the current region of interest in the selected ultrasound image frame.

13. The method of claim 1 further comprising:
receiving a threshold input; and
selecting the portions of the historical ultrasound image frame to be overlapped with the live ultrasound image based on the threshold input.

14. An apparatus comprising:
a non-transitory computer-readable medium to direct a processor to:
present a live ultrasound image on a display of an ultrasound imaging system;
retrieve a historical ultrasound image frame; and
overlap at least portions of the historical ultrasound image frame with the live ultrasound image.

15. The apparatus of claim 14, wherein at least one of the live ultrasound image and the portions of the historical ultrasound image frame that are being overlapped are outlined or semi-transparent.

16. The apparatus of claim 14, wherein the non-transitory computer-readable medium is to further direct the processor to highlight the portions of the historical ultrasound image frame that overlap with the current ultrasound image.

17. The apparatus of claim 14, wherein the non-transitory computer-readable medium is to further direct the processor to remove portions of the live ultrasound image being overlapped by the historical ultrasound image frame.

18. The apparatus of claim 14, wherein the portions of the live ultrasound image that are removed comprise pixels depicting blood flow.

19. An apparatus comprising:
a non-transitory computer-readable medium to direct a processor to:
retrieve a historical ultrasound image frame;
retrieve stored coordinates of a historical region of interest in the historical ultrasound image frame;
display a selected ultrasound image frame from a live ultrasound image; and
displaying a current region of interest in the selected ultrasound image frame, the current region of interest having coordinates in the selected ultrasound image frame based upon the stored coordinates retrieved from the historical region of interest in the historical ultrasound image frame.

20. The apparatus of claim 19, wherein the non-transitory computer-readable medium is to further direct the processor to:
retrieve stored settings indicating measurement functions associated with the historical region of interest of the historical ultrasound image frame; and
carry out the measurement functions with respect to the current region of interest in the selected ultrasound image frame.
